(19) United States
(12) Patent Application Publication
(10) Pub. No.: US 2021/0277399 A1
(43) Pub. Date: Sep. 9, 2021

(54) Methods of Imaging and Tracking Nucleic Acids in Cells

(71) Applicant: Emory University, Atlanta, GA (US)

(72) Inventors: Tewoderos Ayele, Atlanta, GA (US); Jennifer Heemstra, Atlanta, GA (US)

(21) Appl. No.: 17/172,480

(22) Filed: Feb. 10, 2021

Related U.S. Application Data
(60) Provisional application No. 62/972,270, filed on Feb. 10, 2020.

(51) Int. Cl. C12N 15/115

(52) U.S. Cl. CPC C12N 15/115; C12N 2310/14; C12N 2310/11; C12N 2320/32; C12N 2310/3513; C12N 2310/3519; C12N 2310/16

(57) ABSTRACT

This disclosure relates to methods of imaging and tracking RNA or other nucleic acids in cells. In certain embodiments, this disclosure relates to methods of labeling intracellular nucleic acids by expressing in the cell RNA containing an aptamer sequence that specifically binds a label, e.g., fluorescent label, or placing in the cell nucleic acids containing an aptamer sequence that specifically binds a label, wherein the label comprises a carbene forming group, such as a diazirine group, under conditions such that the label specifically binds the aptamer sequence forming a complex; and exposing the complex to electromagnetic radiation sufficient for carbene formation resulting in a covalent bond to the aptamer sequence and the label.

### CROSS-REFERENCE TO RELATED APPLICATIONS

This application claims the benefit of U.S. Provisional Application No. 62/972,270 filed Feb. 10, 2020. The entirety of this application is hereby incorporated by reference for all purposes.

### STATEMENT REGARDING FEDERALLY SPONSORED RESEARCH OR DEVELOPMENT

This invention was made with government support under GM116991 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 20092US_ST25.txt. The text file is 3 KB, was created on Feb. 10, 2021, and is being submitted electronically via EFS-Web.

### BACKGROUND

Trafficking of messenger RNA (mRNA) to subcellular compartments plays an essential role in RNA homeostasis and cellular function. This spatiotemporal control of mRNA localization is a common characteristic for a significant fraction of transcripts. Fluorescent microscopy has dramatically increased our understanding of the heterogeneity of transcript regulation and the complex subcellular interactions of RNAs and proteins. However, this relies on the ability to fluorescently label cellular RNAs without significantly perturbing their structure or localization. The earliest approaches to fluorescently tagging cellular RNAs utilized probes capable of binding to an RNA of interest (ROI) through Watson-Crick-Franklin base pairing, including fluorescent in situ hybridization (FISH) and molecular beacons. While these methods yielded much of the current day knowledge on RNA localization, they generally require cell fixation, and thus cannot provide insight into trafficking and dynamics of cellular RNAs. Other approaches for visualizing mRNA utilize GFP-fused RNA binding proteins such as MS2, λN, PCP, or Cas proteins. These fluorescently tagged proteins recognize a specific sequence that is incorporated multiple times onto ROI. These methodologies suffer from the fact that the unbound fluorescent protein creates significant background signal. This necessitates functionalization of the ROI with multiple copies of the target RNA sequence, and the size of that sequence as well as the heavy load of the associated proteins can alter the native localization and functional properties of the RNA. Improved methods of imaging and tracking RNA are needed.
Anderson et al. report RNA granules contain various ribosomal subunits, translation factors, decay enzymes, helicases, scaffold proteins, and RNA-binding proteins, and they control the localization, stability, and translation of their RNA cargo. J Cell Biol, 2006, 172: 6, 803-808.
Babendure et al. report aptamers switch on fluorescence of triphenylmethane dyes. JACS, 2003, 125 (48), 14716-14717.
Ayele et al. report fluorogenic photoaffinity labeling of proteins in living cells. Bioconjugate chemistry, 2019, 30 (5), 1309-1313.

References cited herein are not an admission of prior art.

### SUMMARY

This disclosure relates to methods of imaging and tracking RNA or other nucleic acids in cells. In certain embodiments, this disclosure relates to methods of labeling intracellular nucleic acids by expressing in the cell RNA containing an aptamer sequence that specifically binds a label, e.g., fluorescent label, or placing in the cell nucleobase polymers containing an aptamer sequence that specifically binds a label, wherein the label comprises a carbene forming group, such as a diazirine group, under conditions such that the label specifically binds the aptamer sequence forming a complex; and exposing the complex to electromagnetic radiation sufficient for carbene formation resulting in a covalent bond to the aptamer sequence and the label providing a covalently labeled aptamer sequence.

In certain embodiments, the method further comprises the step of producing a fluorescent signal by exposing the covalently labeled aptamer sequence to electromagnetic radiation. In certain embodiments, exposing the covalently labeled aptamer sequence to electromagnetic radiation is exposing the covalently labeled aptamer sequence to ultraviolet light. In certain embodiments, the method further comprises the step of imaging the fluorescent signal. In certain embodiments, imaging the fluorescent signal is observing a fluorescent emission. In certain embodiments, the method further comprises the step of tracking the location of the fluorescent signal.

In certain embodiments, this disclosure relates to methods comprising: a) providing, 1) a conjugate comprising a fluorescent tag linked to a diazirine group and 2) a nucleic acid encoding RNA comprising a first aptamer segment that specifically binds the fluorescent tag and a second segment; b) transfecting or inserting the nucleic acid into a cell such that RNA is expressed in the cell; d) incubating the cell with the conjugate such that the fluorescent tag and the RNA form a complex inside the cell; and e) exposing the complex to ultraviolet radiation such that a covalent bond between the RNA and the conjugate forms providing labeled RNA inside the cell with a fluorescent signal.

In certain embodiments, the second segment is a nucleobase polymer, nucleic acid, or RNA that specifically binds to a protein in the cell.

In certain embodiments, the first aptamer segment comprises or has a 5′ end consisting of the nucleic acid sequence of GGATCCCGACTGGCGAGAGCCAGGTAACGAATGGATCC (SEQ ID NO: 1).

In certain embodiments, the conjugate is a salt of N-(4-(4-(dimethylamino)phenyl)(4-(3-(3-methyl-3H-diazirin-3-yl)propanamido)phenyl)methylene)cyclohexa-2,5-dien-1-ylidene)-N-methylmethanaminium (MGD2) or derivative thereof.

In certain embodiments, exposing the complex to ultraviolet radiation is irradiation at 365 nm. In certain embodiments, exposing the complex to ultraviolet radiation is for not more than 15 min when the conjugate is at a concentration of 30 μM. In certain embodiments, the exposing the complex to ultraviolet radiation is for not more than 10 min when the conjugate is at a concentration of 100 μM.

In certain embodiments, the methods further comprise the step of producing a fluorescent signal by exposing the covalently labeled aptamer sequence to electromagnetic radiation. In certain embodiments, exposing the covalently labeled aptamer sequence to electromagnetic radiation is exposing the covalently labeled aptamer sequence to ultraviolet light with an excitation at 625 nm.

In certain embodiments, the methods further comprise the step of imaging the fluorescent signal. In certain embodiments, imaging the fluorescent signal is observing a fluorescent emission at 660 nm. In certain embodiments, the methods further comprise the step of tracking the location of the fluorescent signal.

In certain embodiments, this disclosure relates to methods comprising transfecting or inserting into a cell a nucleic acid that encodes RNA under conditions that the RNA is expressed, or transfecting or inserting into a cell RNA or a nucleic acid, wherein the RNA or nucleic acid contains an aptamer sequence that specifically binds a label comprises a carbene forming group, such as a diazirine group; exposing the cell to the label such that the label enters the cell, or directly inserting the label into the cell, under conditions such that the label specifically binds the aptamer sequence inside the cell forming a complex; exposing the complex to electromagnetic radiation sufficient for carbene formation resulting in a covalent bond to the aptamer sequence; and imaging or tracking the label in the cell. In certain embodiments, the label is a fluorescent tag.

In certain embodiments, the conjugate is a salt of N-(4-((4-(dimethylamino)phenyl)(4-(3-(3-methyl-3H-diazirin-3-yl)propanamido)phenyl)methylene)cyclohexa-2,5-dien-1-ylidene)-N-methylmethanaminium (MGD2) or derivative thereof.

In certain embodiments, the nucleic acid encoding RNA comprising a first aptamer segment that specifically binds the fluorescent tag comprises or has a 5′-end consisting of the nucleic acid sequence GGATCCCGACTGGCGAGAGCCAGGTAACGAATGGATCC (SEQ ID NO: 1).

In certain embodiments, the methods further comprise the step of imaging the fluorescent signal. In certain embodiments, the methods further comprise the step of tracking the location of the fluorescent signal. In certain embodiments, the second segment specifically binds to a protein in the cell. In certain embodiments, the protein is a granule. In certain embodiments, the second segment specifically binds to a nucleic acid (RNA/DNA) in the cell.

### BRIEF DESCRIPTION OF THE SEVERAL VIEWS OF THE DRAWINGS

FIG. 1A is a schematic representation of fluorogenic photoaffinity labeling of MGA-functionalized mRNA. The MGA-functionalized mRNA binds to the fluorogenic dye and induces fluorescence enhancement. UV-irradiation results in covalent attachment of the dye to the RNA of interest.

FIG. 1B illustrates the structures of MG and MGD2. The canonical MG molecule was functionalized with a diazirine linker to enable photoaffinity labeling of MGA.

FIG. 1C shows data on emission (solid) and excitation (dashed) spectra of MGD2 bound to 1×MGA-mGFP.

FIG. 1D shows data on fluorescence of MGD2, 1×MGA-mGFP, and 6×MGA-mGFP in 1×PBS.

FIG. 1E shows data using UV-irradiated for different lengths of time.

FIG. 2A shows a schematic representation of MGA-functionalized mRNA constructs.

FIG. 2B shows fluorescence intensity of RNA foci in untransfected Neuro-2a cells or Neuro-2a cells expressing mCDK6 functionalized with 1×MGA or 6×MGA at the 5′ end. (n=6 foci for no transfection and transfection with control RNA, n=50 foci for 1×MGA and 6×MGA). Box plots show median, upper and lower quartiles, whiskers extending to 5th and 95th percentile, and mean represented by a cross sign.

FIG. 2C shows representative normalized line-scan of colocalized FISH (line) and MGA-mCDK6 (line) labeling with MGD2.

FIG. 2D shows representative normalized line-scan of RNA granules detected by MGA-functionalized mCDK6 (line) but not with FISH (line).

FIG. 3A shows data on fluorescence signal comparison of MGD2-labeled mRNA vs GFP-labeled G3BP1 protein.

FIG. 3B shows line-scan data of relative intensity of lines from images showing phase separation and RNA granule maturation. Image slices were taken every 2.8 min

FIG. 3C shows image relative intensity line-scan data indicating disappearance of RNA granule with 5% 1,6-hexandiol treatment.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The term “comprising” in reference to a nucleic acid having a nucleotide sequence refers a nucleotide that may contain additional 5′-end or 3′-end nucleotides, i.e., the term is intended to include the nucleic acid sequence within a larger sequence. The term “consisting of in reference to a nucleic acid having a nucleotide sequence refers a nucleic acid having the exact number of nucleotides in the sequence and not more or having not more than a rage of nucleotides expressly specified in the claim. In certain embodiments, the disclosure contemplates that the “5′-end” of a nucleic acid may consist of an nucleotide sequence,” which refers to the 5′-end of the nucleic acid having the exact number of nucleotides in the sequence and not more or having not more than a rage of nucleotides specified in the claim however the 3′-end may be connected to additional nucleotides, e.g., as part of a larger nucleic acid.

An “aptamer” refers to a nucleobase polymer or nucleic acid sequence, e.g., DNA/RNA or combination thereof, that acts as a receptor which specifically binds a ligand such as a fluorescent tag. Aptamers typically contain secondary structures, e.g., one or more loops, bends, or hairpins, as a result of incomplete base pairing.

The term “inserting” into a cell refers to the process of introducing nucleobase polymers, nucleic acids, DNA, or RNA into the cytosol/cytoplasm of cells e.g., eukaryotic somatic cells, bypassing the cellular membrane. Phosphate backbones of DNA and RNA are negatively charged molecules and cell membranes are negatively charged. Thus, nucleic acids typically do not spontaneously pass through cellular membranes. A variety of techniques are known in the art to move extracellular nucleic acid inside cells. Inserting nucleic acids into cells can be accomplished by mechanical means, e.g., microneedles/microinjection, electroporation, chemical, or biomolecular means, e.g., surrounding the nucleic acid in recombinant viral particles that release the interior components after cellular fusion and entry.

Cellular “transfection” refers to is the process of introducing nucleic acids into cells by chemical mechanism that interact with bilayer membranes. For example, calcium phosphate and diethylaminoethyl (DEAE)-dextran and cationic lipid-based reagents are able to coat nucleic acids, enabling the complexes of DNA:transfection reagents to cross cell membranes These complexes may be integrated into lipids formations/artificial liposomes. Cationic lipids are typically mixed with neutral lipids such as L-dioleoyl phosphatidylethanolamine to enhance fusion with lipid bilayers.

As used herein, the term “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a proteinaceous compound that has a molecular weight of greater than 1,000, 2,000 or 5,000.

The term “specific binding agent” refers to a molecule, such as a protein, antibody, or aptamer, that binds a target molecule with a greater affinity than other random molecules, proteins, or nucleic acids. Examples of specific binding agents include antibodies that bind an epitope of an antigen or a receptor which binds a ligand. “Specifically binds” refers to the ability of a specific binding agent (such as an ligand, receptor, enzyme, aptamer, antibody or binding region/fragment thereof) to recognize and bind a target molecule such that its affinity (as determined by, e.g., affinity ELISA or other assays) is at least 10 times as great, but optionally 50 times as great, 100, 250 or 500 times as great, or even at least 1000 times as great or more as the affinity of the same for any other random molecule, nucleic acid, or polypeptide.

A “diazirine” or “3H-diazirene” refers to organic molecules having a carbon bound to two nitrogen atoms, which are double-bonded to each other, forming a heterocyclic ring. Upon irradiation with ultraviolet light, diazirines form reactive carbenes, which can form covalent bonds with C—H, N—H, and O—H groups. Hill et al. report a variety of diazirine photoaffinity probes. J. Med. Chem. 2018, 61, 6945-6963.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon to carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of covalent molecular arrangements that can be used to bridge to molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups and alkoxyalkyl groups.

A “fluorescent tag” or “fluorescent dye” refers to a compound that can re-emit electromagnetic radiation upon excitation with electromagnetic radiation (e.g. ultraviolet light) of a different wavelength. Typically, the emitted light has a longer wavelength (e.g. in visible spectrum) than the absorbed radiation. As the emitted light typically occurs almost simultaneously, i.e., in less than one second, when the absorbed radiation is in the invisible ultraviolet region of the spectrum, the emitted light may be in the visible region resulting in a distinctive identifiable color signal. Small molecule fluorescent tags typically contain several combined aromatic groups, or planar or cyclic molecules with multiple interconnected double bonds. Chen et al. report a variety of fluorescent tags that can be viewed across the visible spectrum. Nature Biotechnology, 2019, 37, 1287-1293. The term “fluorescent tag” is intended to include compounds of larger molecular weight such as natural fluorescent proteins, e.g., green fluorescent protein (GFP) and phycobiliproteins (PE, APC), and fluorescence particles such as quantum dots, e.g., preferably having 2-10 nm diameter.

As used herein, the term “small molecule” refers to any variety of covalently bound molecules with a molecular weight of less than 900 or 1000. Typically, the majority of atoms include carbon, hydrogen, oxygen, nitrogen, sulfur and halogens.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen, hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

The term “nucleobase polymer” refers to a polymer comprising nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may contain DNA or RNA or a combination of DNA or RNA nucleotides or may be single or double stranded or both, e.g., they may contain overhangs, hairpins, bends, etc. Nucleobase polymers may contain naturally occurring or synthetically modified bases and backbones.

With regard to the nucleobases, it is contemplated that the term encompasses isobases, otherwise known as modified bases, e.g., are isoelectronic or have other substitutes configured to mimic naturally occurring hydrogen bonding base-pairs. Examples of nucleotides with modified adenosine or guanosine include, but are not limited to, hypoxanthine, xanthine, 7-methylguanine. Examples of nucleotides with modified cytidine, thymidine, or uridine include 5,6-dihydrouracil, 5-methylcytosine, 5-hydroxymethylcytosine. Contemplated isobases include 2′-deoxy-5-methylisocytidine (iC) and 2′-deoxy-isoguanosine (iG) (see U.S. Pat. Nos. 6,001,983; 6,037,120; 6,617,106; and 6,977,161). Within any of the sequences disclosed herein U may be substituted for T, or T may be substituted for U. U is one of the four nucleobases in the nucleic acid RNA. In DNA, the uracil (U) nucleobase is replaced by thymine (T). Uracil is a demethylated form of thymine. Thus, from a structural standpoint natural RNA is distinct from DNA due to the presence of a 2′ hydroxy on the ribose unit and demethylated thymine bases.

Nucleobase polymers may be chemically modified, e.g., within the sugar backbone or on the 5′ or 3′ ends. The nucleobase polymers can be modified, for example, 2′-amino, 2′-O-allyl, 2′-fluoro, 2′-O-methyl, 2′-methyl, 2′-H of the ribose ring. In certain embodiments, nucleobase polymers disclosed herein may contain monomers of phosphodiester, phosphorothioate, methylphosphonate, phosphorodiamidate, piperazine phosphorodiamidate, ribose, 2′-O-methy ribose, 2′-O-methoxyethyl ribose, 2′-methyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphonamidate, morpholin-2-ylmethanol, (2-(hydroxymethyl)morpholino) (piperazin-1-yl)phosphinate, or peptide nucleic acids or combinations thereof. In certain embodiments, nucleobase polymers are contemplated to comprise phosphorodiamidate morpholino oligomers (PMO). In certain embodiments, the nucleobase polymer comprises monomers of (2-(hydroxymethyl)morpholino)(piperazin-1-yl)phosphinate.

Imaging and Tracking mRNA in Live Mammalian Cells Via Fluorogenic Photoaffinity Labeling

Studying intracellular trafficking of RNA which bind to granules by existing imaging methods is challenging due to background signals. In order to develop a broadly applicable strategy that can be implemented for both fixed cell and live cell imaging and that would enable robust labeling with only a single small RNA fusion and fluorophore tag, a fluorescent probe and an RNA of interest (ROI) were covalently tethered. This smaller RNA fusion can be used in imaging methods to increase signal-to-background ratios. Using a photoaffinity labeling approach provides temporal control over the RNA labeling process.

Malachite green was modified to incorporate a photo-reactive diazirine linker, which allows for covalent labeling of its cognate aptamer upon irradiation with UV light. By placing this aptamer at the 5′ end of the mRNA, target-specific fluorescence enhancement and labeling of the ROI was achieved. Fixed cell imaging of aptamer-functionalized mRNA showed formation of RNA stress granules in response to arsenite exposure. Compared to hybridization-based RNA labeling, enhanced sensitivity and lower background signal was obtained with the MGD2/MGA system. Furthermore, the dynamics of RNA granules containing a single aptamer-functionalized ROI can be tracked in live cells upon covalent attachment of the fluorogenic probe.

This strategy provides several advantages for RNA imaging applications. First, the far red-shifted fluorescence emission wavelength and the fluorogenic nature of the MGD2 dye allows for minimal background signal generated from cellular autofluorescence and unbound small molecules. Second, the temporally controlled covalent attachment of the fluorogen to its cognate aptamer enables the labeling to withstand washout steps and allows tracking of RNAs labeled during a specific time window, a feature which is necessary for pulse-chase studies and other experiments that require media exchange. Third, a single aptamer fusion of 57 nt was sufficient to image RNA granules in both live and fixed cells. This is significantly smaller than the fusions required in other RNA labeling approaches, which typically append numerous copies of the respective tag and fluorescent probe or proteins, producing a fusion that can add thousands of kDa. This strategy is contemplated to be generalizable enabling the development of additional aptamer-photoaffinity probe combinations for multiplexed and multicolor imaging.

### Methods of Use

This disclosure relates to methods of imaging and tracking RNA or other nucleic acids in cells. In certain embodiments, this disclosure relates to methods of labeling intracellular nucleic acids by expressing in the cell RNA containing an aptamer sequence that specifically binds a label, e.g., fluorescent label, or placing in the cell a nucleobase polymer containing an aptamer sequence that specifically binds a label, wherein the label comprises a carbene forming group, such as a diazirine group, under conditions such that the label specifically binds the aptamer sequence forming a complex; and exposing the complex to electromagnetic radiation sufficient for carbene formation resulting in a covalent bond to the aptamer sequence and the label.

In certain embodiments, the methods further comprise the step of producing a fluorescent signal by exposing the covalently labeled aptamer sequence to electromagnetic radiation. In certain embodiments, exposing the covalently labeled aptamer sequence to electromagnetic radiation is exposing the covalently labeled aptamer sequence to ultraviolet light. In certain embodiments, the methods further comprise the step of imaging the fluorescent signal. In certain embodiments, imaging the fluorescent signal is observing or detecting a fluorescent emission. In certain embodiments, the method further comprises the step of tracking the location of the fluorescent signal.

In certain embodiments, this disclosure relates to methods comprising: a) providing, 1) a conjugate comprising a fluorescent tag linked to a diazirine group and 2) a nucleic acid encoding RNA comprising a first aptamer segment that specifically binds the fluorescent tag and a second segment; b) transfecting or inserting the nucleic acid into a cell such that RNA is expressed in the cell; d) incubating the cell with the conjugate such that the fluorescent tag and the RNA form a complex inside the cell; and e) exposing the complex to ultraviolet radiation such that a covalent bond between the RNA and the conjugate forms providing labeled RNA inside the cell with a fluorescent signal.

In certain embodiments, the second segment is a nucleobase polymer, nucleic acid, DNA or RNA that specifically binds to a protein in the cell.

In certain embodiments, the first aptamer segment comprises or has a 5′ end consisting of the nucleic acid sequence of GGATCCCGACTGGCGAGAGCCAGGTAACGAATGGATCC (SEQ ID NO: 1) or label binding/fluorescent dye bind variant thereof.

In certain embodiments, the conjugate is a salt of N-(4-((4-(dimethylamino)phenyl)(4-(3-(3-methyl-3H-diazirin-3-yl)propanamido)phenyl)methylene)cyclohexa-2,5-dien-1-ylidene)-N-methylmethanaminium (MGD2) or derivative thereof.

In certain embodiments, the exposing the complex to ultraviolet radiation is irradiation at 365 nm. In certain embodiments, the exposing the complex to ultraviolet radiation is exposing the complex to ultraviolet radiation is for not more than 15 min when the conjugate is at a concentration of 30 μM. In certain embodiments, the exposing the complex to ultraviolet radiation is exposing the complex to ultraviolet radiation is for not more than 10 min when the conjugate is at a concentration of 100 μM.

In certain embodiments, the methods further comprise the step of producing a fluorescent signal by exposing the covalently labeled aptamer sequence to electromagnetic radiation. In certain embodiments, exposing the covalently labeled aptamer sequence to electromagnetic radiation is exposing the covalently labeled aptamer sequence to ultraviolet light with an excitation at 625 nm.

In certain embodiments, the methods further comprise the step of imaging the fluorescent signal. In certain embodiments, imaging the fluorescent signal is detecting, measuring or observing a fluorescent emission at 660 nm. In certain embodiments, the methods further comprise the step of tracking the location of the fluorescent signal.

In certain embodiments, this disclosure relates to methods comprising transfecting, or inserting into a cell, a nucleic acid that encodes RNA under conditions that the RNA is expressed, or transfecting or inserting into, a cell RNA or a nucleic acid, wherein the RNA or other nucleic acid contains an aptamer sequence that specifically binds a label comprising a carbene forming group, such as a diazirine group; exposing the cell to the label such that the label enters the cell, or directly inserting the label into the cell, under conditions such that the label specifically binds the aptamer sequence forming a complex; exposing the complex to electromagnetic radiation sufficient for carbene formation resulting in a covalent bond to the aptamer sequence; and imaging or tracking the label in the cell. In certain embodiments, the label is a fluorescent tag.

In certain embodiments, this disclosure relates to methods comprising: a) providing, 1) a conjugate comprising a fluorescent tag linked to a diazirine group and 2) a nucleic acid encoding RNA comprising a first aptamer segment that specifically binds the fluorescent tag and a second segment; b) transfecting or inserting the nucleic acid into a cell under conditions such that RNA is expressed in the cell; d) incubating or inserting in the cell the conjugate under conditions such that the fluorescent tag and the RNA form a complex inside the cell; and e) exposing the complex to ultraviolet radiation such that a covalent bond between the RNA and the conjugate forms providing labeled RNA inside the cell with a fluorescent signal.

In certain embodiments, the conjugate is a salt of N-(4-((4-(dimethylamino)phenyl)(4-(3-(3-methyl-3H-diazirin-3-yl)propanamido)phenyl)methylene)cyclohexa-2,5-dien-1-ylidene)-N-methylmethanaminium (MGD2). In certain embodiments, the nucleic acid encoding RNA comprising a first aptamer segment that specifically binds the fluorescent tag comprises or has a 5′-end consisting the nucleic acid sequence GGATCCCGACTGGCGAGAGCCAGGT AACGAATGGATCC (SEQ ID NO: 1).

In certain embodiments, the methods further comprise the step of imaging the fluorescent signal. In certain embodiments, the methods further comprise the step of tracking the location of the fluorescent signal. In certain embodiments, the second segment specifically binds to a protein in the cell. In certain embodiments, the second segment specifically binds or hybridizes to a nucleic acid (RNA/DNA) in the cell. In certain embodiments, the protein is a granule. In certain embodiments, exposing the complex to ultraviolet radiation such that a covalent bond between the RNA and the conjugate forms is irradiation at 365 nm.

In certain embodiments, this disclosure relates to methods comprising: a) providing, 1) a conjugate comprising a fluorescent tag linked to a diazirine group and 2) RNA comprising a first aptamer segment that specifically binds the fluorescent tag and a second segment; b) transfecting or placing the RNA into the cell; d) incubating the cell with the conjugate under conditions such that the fluorescent tag and the RNA form a complex inside the cell; and e) exposing the complex to ultraviolet radiation such that a covalent bond between the RNA and the conjugate forms providing labeled RNA inside the cell with a fluorescent signal.

In certain embodiments, the RNA comprising a first aptamer segment that specifically binds the fluorescent tag comprises or has a 5′-end consisting the nucleic acid sequence GGATCCCGACTGGCGAGAGCCAGGTAACGAATGGATCC (SEQ ID NO: 1).

In certain embodiments, the methods further comprise the step of imaging the fluorescent signal. In certain embodiments, the methods further comprise the step of tracking the location of the fluorescent signal. In certain embodiments, the second segment specifically binds to a protein in the cell. In certain embodiments, the second segment specifically binds to a nucleic acid (RNA/DNA) in the cell. In certain embodiments, the protein is a granule. In certain embodiments, the conjugate is a salt of N-(4-((4-(dimethylamino)phenyl)(4-(3-(3-methyl-3H-diazirin-3-yl)propanamido)phenyl)methylene)cyclohexa-2,5-dien-1-ylidene)-N-methylmethanaminium. In certain embodiments, exposing the complex to ultraviolet radiation such that a covalent bond between the RNA and the conjugate forms is irradiation at 365 nm.

In certain embodiments, this disclosure relates to methods comprising: a) providing, 1) a conjugate comprising a fluorescent tag linked to a diazirine group and 2) a nucleobase polymer comprising a first aptamer segment that specifically binds the fluorescent tag and a second segment; b) transfecting or placing the nucleobase polymer into the cell; d) incubating the cell with the conjugate under conditions such that the fluorescent tag and the nucleobase polymer form a complex inside the cell; and e) exposing the complex to ultraviolet radiation such that a covalent bond between the nucleobase polymer and the conjugate forms providing labeled nucleobase polymer inside the cell with a fluorescent signal.

In certain embodiments, the nucleobase polymer comprising a first aptamer segment that specifically binds the fluorescent tag comprises or has a 5′-end consisting the nucleic acid sequence GGATCCCGACTGGCGAGAGCCAGGTAACGAATGGATCC (SEQ ID NO: 1) wherein all Ts are Us or wherein individually and independently at all occurrences T is optionally U.

In certain embodiments, methods include the step of detecting, measuring, imaging, or tracking which entails recording measurement data, imaging data, or tracking data on non-transitory computer readable medium.

### EXAMPLES

### Synthesis of N-(4-(bis(4-(dimethylamino)phenyl)methyl)phenyl)-3-(3-methyl-3H-diazirin-3-yl)propanamide (Leucomalachite Green Diazirine)

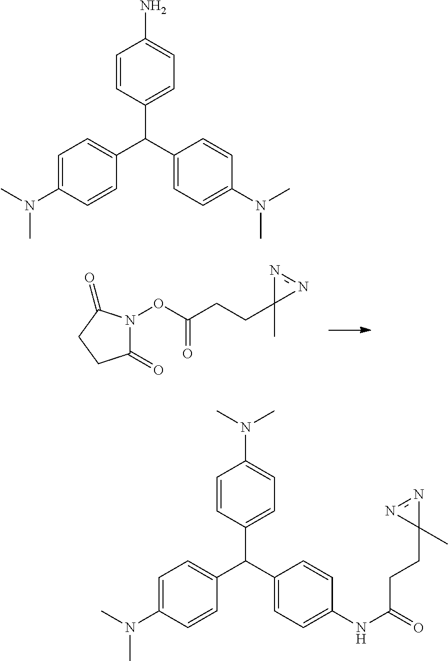

In an oven dried 5 mL flask, p-amino-leucomalachite green (25.0 mg, 72.7 μmol) [prepared as provided in Xing et al., Journal of the Science of Food and Agriculture 2009, 89 (13), 2165-2173] was dissolved in 1 mL of dry pyridine under inert gas. To this solution, 1.2 eq. of NHS-diazirine (19.6 mg, 86.83 μmol) was added and the solution was stirred at room temperature overnight. The reaction was then concentrated under reduced pressure to obtain dark green oil. The crude oil was dissolved in minimal amount of methanol and loaded on a preparative TLC with 30% EtOAc/MeOH as a mobile phase. The product band was scraped off from the preparative TLC and the product was filtered from the silica using MeOH and dried under reduced pressure. Yield=11.6 mg, 35.31%. ¹H NMR (500 MHz, DMSO) δ 0.99 (s, 3H), 1.64 (t, 2H, J=7.57 Hz), 2.20 (t, 2H, J=7.3 Hz), 3.04 (s, 12H), 5.63 (s, 1H), 7.03 (d, 2H, J=8.3 Hz), 7.24 (d, 4H, J=7.3), 7.52-7.69 (m, 6H), 10.15 (s, 1H). ¹³C NMR (500 MHz, DMSO) δ 19.36, 25.76, 29.53, 30.69, 44.52, 53.93, 119.21, 129.06, 130.14, 137.68, 169.75. LRMS (ESI-TOP) m z Calcd for C₂₈H₃₃N₅O [M+H]⁺ 456.2758; Found 456.2746.

### Synthesis of N-(4-((4-(dimethylamino)phenyl)(4-(3-(3-methyl-3H-diazirin-3-yl) Propan amido)phenyl)methylene)cyclohexa-2,5-dien-1-ylidene)-N-methylmethanaminium Chloride (Malachite Green Diazirine, MGD2)

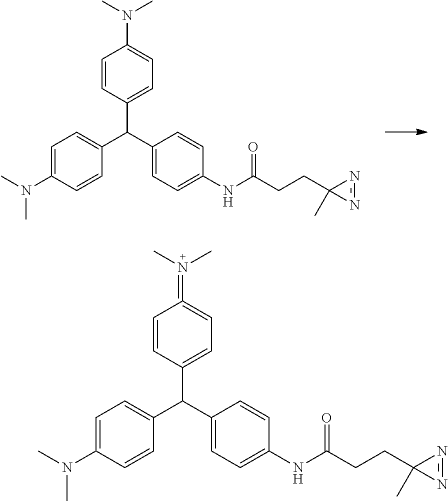

11.64 grams (25.55 μmol) of leucomalachite green diazirine was dissolved in 20% MeOH/EtOAc. To this solution, 1.2 eq. of chloranil (7.54 mg, 30.66 μmol) was added and the solution was stirred at room temperature for 3 h. The dark green solution was concentrated under reduced pressure and flash column purified using EtOAc to remove excess chloranil. Then mobile phase was then switched to 50% MeOH/DCM to isolate the crude product. The collected crude product was concentrated under reduced pressure and further purified by preparative TLC using 20% MeOH/DCM as mobile phase. Yield=10.30 mg, 88.68%. ¹H NMR (500 MHz, DMSO) δ 1.03 (s, 3H), 1.69 (t, 2H, J=7.1 Hz), 2.37 (t, 2H, J=7.3 Hz), 3.14 (s, 6H), 3.27 (s, 6H), 7.07 (d, 4H, J=8.8 Hz), 7.31 (t, 6H, J=9.3 Hz), 7.94 (d, 2H, J=8.3 Hz). ¹³C NMR (500 MHz, DMSO) δ 19.35, 25.70, 28.93, 29.37, 40.39, 48.50, 113.63, 118.52, 126.24, 133.20, 136.25, 140.06, 156.23. LRMS (ESI-TOP) m z Calcd. for C₂₈H₃₂N₅O⁺ [M]⁺ 454.2601; Found 454.259

### MGA Array Plasmid Construction

As reported in Yerramilli & Kim, ACS Synth. Biol. 2018, 7, 758-766, the DNA sequence of the MGA core unit is GGATCCCGACTGGCGAGAGCCAGGTAACGAATGGATCC (SEQ ID NO: 1) and MGA6 is TCTAGATGGTGTTTTGGTTTGGTCAACGGATCCCGA CTGGCGAGAGCCAGGTAACGAATGGATCCGTTGACACCCAACAAAAAAAAACACG GATCCCGACTGGCGAGAGCCAGGTAACGAATGGATCCGTGTAAAAAAAAACCC AAAAAGCCGGGATCCCGACTGGCGAGAGCCAGGTAACGAATGGATCCCGGCAA CCAACCAACCAACCAAAAAGGATCCCGACTGGCGAGAGCCAGGTAACGAATGG ATCCTTTTTCCCCAGACAACGAGAGTCTTGGATCCCGACTGGCGAGAGCCAGGTA ACGAATGGATCCAAGAGAGAGACCACATGCAAAATCTGGATCCCGACTGGCGAG AGCCAGGTAACGAATGGATCCAGATGCTAGCGCGGATCCGAATTCGAGCTCCGTC GACAAGCTTT (SEQ ID NO: 2).

Single copy malachite green aptamer-containing plasmids were derived from pcDNA3.1(+), digested with BamHI and NotI and similarly cut acGFP from pAcGFP-N1-SialT (Addgene plasmid #87324) was inserted to create pcDNA3.1-acGFP. pcDNA3.1-acGPF was digested with AgeI and XbaI and inserted a similarly digested PCR product of mCDK6 from pcDNA3.1 mouse cdk6 wt (Addgene plasmid #75170). Amplification was achieved using 5′-ATATATACCGGTACCATGGAGAAGGACAGCCT-3′ (SEQ ID NO: 3) and 5′-ATATATTCTAGAATCAGGCTGTGTTCAGCTCC-3′ (SEQ ID NO: 4), resulting in pcDNA3.1-mouse Cdk6 wt. The single copy malachite green aptamer was inserted by digesting pcDNA3.1acGFP with NheI and BamHI and inserting an annealed oligo pair, 5′-CTAGCGGATCCCGACTGGCGAGAGCCAGGTAACGAATGGATCC-3′ (SEQ ID NO: 5) and 5′-GATCCGGATCCATTCGTTACCTGGCTCTCGCCAGTCGGGATCC-3′ (SEQ ID NO: 6) with compatible overhangs. The resulting vector, pcDNA3.1-1×MGA-acGFP was digested with AgeI and XbaI and inserted a similarly digested PCR product of mCDK6 from pcDNA3.1 mouse cdk6 wt. Amplification was achieved using 5′-ATATATACCGGTACCATGGAGAAGGACAGCCT-3′(SEQ ID NO: 3) and 5′-ATATATTCTAGAATCAGGCTGTGTTCAGCTCC-3′, (SEQ ID NO: 4), resulting in pcDNA3.1-1×MGA-mouse Cdk6 wt. pcDNA3.1-6×MGA-acGFP was made by digesting pcDNA3.1acGFP with NheI and AflII and inserting similarly cut 6×MGA PCR amplified from pUC57-6×MGA with 5′-ATATATGCTAGCTAGATGGTGTTTTGGTTTGG-3′(SEQ ID NO: 7) and 5′-ATATATCTTAAGCGAATTCGGATCCGCG-3′ (SEQ ID NO: 8). pcDNA3.1-6×MGA-mouse Cdk6 was made by digesting pcDNA3.1-6×MGA-acGFP with AgeI and XbaI and inserted a similarly digested PCR product of mCDK6 from pcDNA3.1 mouse cdk6 wt. Amplification was achieved using 5′-ATATATACCGGTACCATGGAGAAGGACAGCCT-3′ (SEQ ID NO: 3) and 5′-ATATATTCTAGAATCAGGCTGTGTTCAGCTCC-3′ (SEQ ID NO: 4), resulting in pcDNA3.1-6×MGA-mouse Cdk6.

### Labeled Photo Crosslinked Aptamer Fused to an RNA of Interest (ROI)

Malachite green aptamer (MGA) was utilized for linking the label, MG. MGA binds to the MG and induces significant red-shifted fluorescence enhancement. The excitation and emission maxima for the MG are also located in the far-red region of the UV spectrum, averting the inherent challenges associated with cellular auto-fluorescence and making this aptamer-ligand pair exceptionally well-suited for live cell imaging.

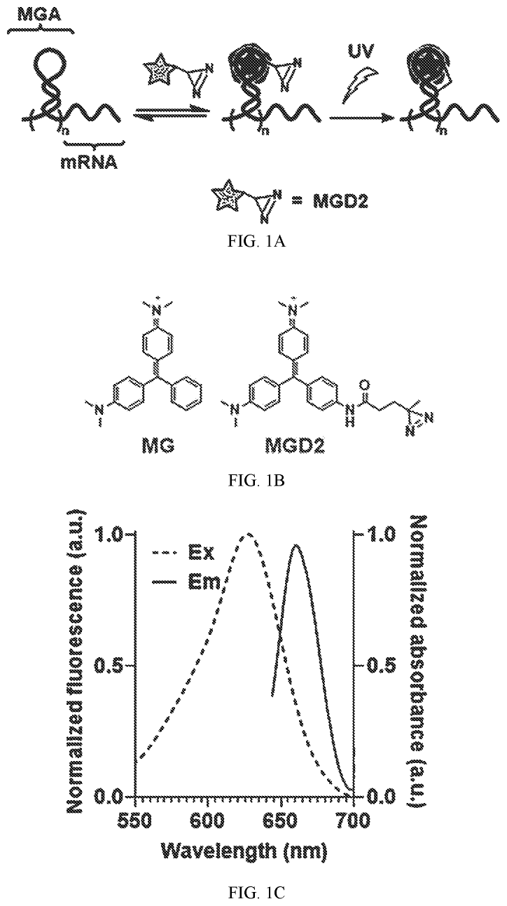

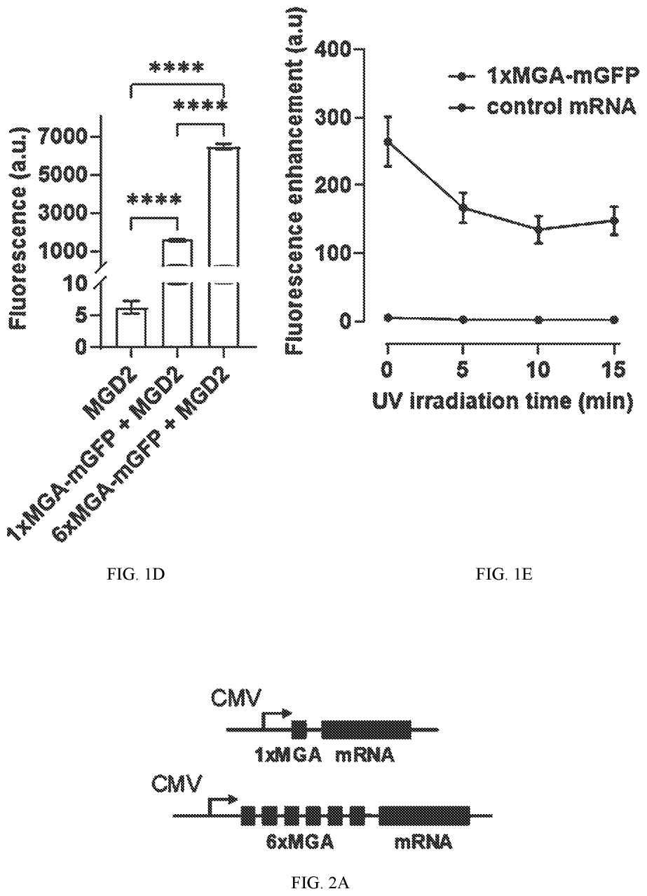

To covalently label the target RNA, the aptamer can be fused to an RNA of interest (ROI), expressed in cells, and the cells incubated with the MG ligand having a photoactivatable handle. UV-irradiation converts the non-covalent binding interaction into a covalent linkage resulting in robust and temporally controlled labeling of the ROI (FIG. 1A). To create a photo-reactive fluorogen, a diazirine linker was appended to the dye (FIG. 1B) providing the MG derivative malachite green diazirine-2 (MGD2). Upon UV-A irradiation at 365 nm, the diazirine linker is activated and produces a carbene moiety that readily reacts with nearby C—H and heteroatom-H bonds. UV-C (254 nm) irradiation has been used for cross-linking RNA-protein interactions by taking advantage of the photo-responsiveness of natural amino acids such as Cys, Lys, Phe, Trp, and Tyr. However, the longer wavelength of 365 nm used to activate MGD2 was done to avoid unwanted cross-linkage of RNA with cellular proteins.

An mRNA of interest can be labeled and imaged in both fixed and live cells using a single 57 nt fusion. This is significantly smaller than the fusions required in other aptamer-based methods, minimizing perturbation of RNA structure and localization. Covalent labeling enables RNA visualization under conditions where the non-covalent systems fail. The dynamics of RNAs was monitored in stress granules. The added robustness and temporal control achieved using this approach advances RNA imaging capabilities and RNA trafficking in biological processes.

### Determination of MGD2 Selectivity in Cellular RNA

To determine the selectivity of MGD2 labeling, 6MGA-mGFP (200 ng) was spiked in into cellular RNA extracted from HeLa cells (400 ng) in 1×PBS. To this solution, MGD2 (50 μM final concentration) was added and the solution was incubated for 20 min at room temperature. After 10 min of UV irradiation, the sample was mixed with equal volume of 50% glycerol solution and heated to 70° C. to denature the RNA. This sample was loaded on to 1% agarose gel containing 1% v/v Clorox® bleach2 and Sybr Gold™. After running the gel for 1 h at a constant 100 V, the gel was frozen with dry ice for 10 min and imaged using GE Amersham Typhoon™. This freezing step enhanced the fluorescence output of the malachite green molecule. Analysis of this gel indicates that the MGD2 selectively labeled the aptamer functionalized mRNA and did not have any detectable labeling of cellular RNA.

In Vitro Studies of Aptamer when Functionalized to an mRNA.

AcGFP mRNA appended with one or six copies of the aptamer sequence at the 5′ end, referred to as 1×MGA-mGFP and 6×MGA-mGFP, respectively, was transcribed. Both MGA-functionalized mRNAs displayed a well-defined absorbance and fluorescence profile with an excitation maximum at 625 nm and an emission maximum 660 nm in the presence of MGD2 (FIG. 1C). Prior to UV-irradiation, fluorescence measurements revealed a 251-fold fluorescence enhancement for MGD2 bound to 1×MGA-mGFP and >1000-fold fluorescence enhancement for MGD2 bound to 6×MGA-mGFP (FIG. 1D).

TABLE 1
Tabular data fluorescence output of MGA array

| | MGD2 | 1xMGA-mGFP + MGD2 | 6xMGA-mGFP + MGD2 |
|---|---|---|---|
| mean | 6.25 | 1570 | 6454 |
| stdev | 0.96 | 73 | 150 |

The effect of UV-irradiation on the fluorescence enhancement was investigated. For this experiment, 1×MGA-mGFP and a control mGFP mRNA that does not contain the aptamer sequence were used. Up to 15 minutes of UV-irradiation did not result in any detectable fluorescence enhancement of MGD2 in the presence of the control mRNA. However, the fluorescence enhancement of 1×MGA-mGFP declined slightly over time and stabilized at approximately 140-fold after 15 minutes of UV irradiation indicating that a single copy of MGA could produce a detectable and stable signal-to-background ratio for cellular imaging experiments.

Labeling specificity was investigated using denaturing PAGE analysis. Both the control GFP mRNA lacking MGA (4 μM) and 1×MGA-GFP mRNA (4 μM) were incubated with MGD2 and UV-irradiated for different lengths of time. Non-specific labeling of the control mRNA was observed after 15 minutes of irradiation in the presence of 30 μM MGD2 and after 10 minutes of irradiation in the presence of 100 μM MGD2. These data were somewhat surprising given the lack of fluorescence enhancement observed for the control RNA in the previous experiment. Thus, this approach does result in some non-specific RNA labeling.

Experiments were performed to determine whether this would create problematic background signal during imaging. The selective reaction of MGD2 (30 μM) with 6×MGA-mGFP was tested in the presence of cellular RNA After 10 min of UV irradiation, bands in the MG channel corresponding to the target RNA and slight impurities we observed, but no bands from labeling of other cellular RNAs. The collective observations from these experiments served as guidelines for irradiation time and dye concentrations used in subsequent cellular labeling experiments.

### Fixed-Cell Imaging of RNA

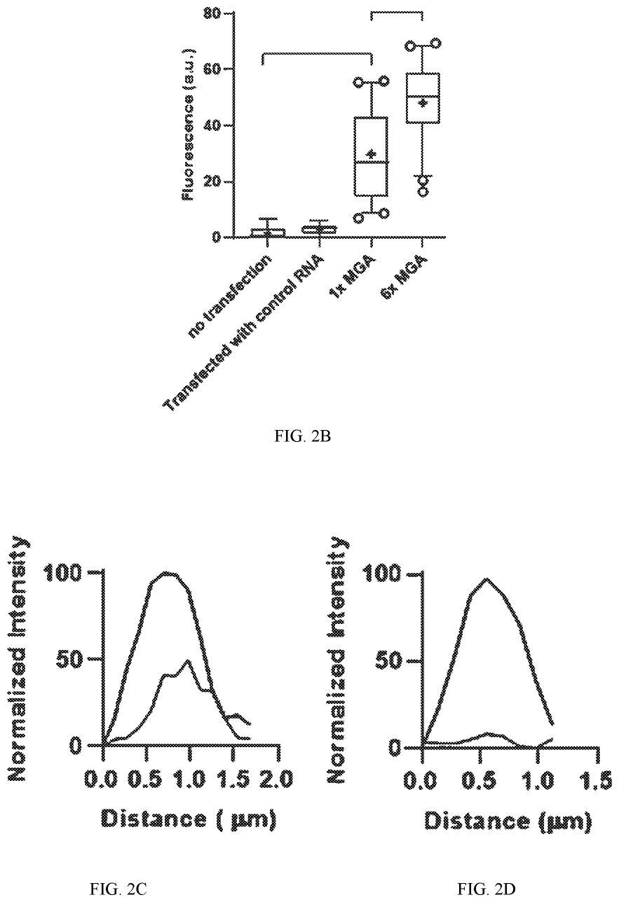

Fixed cell experiments enable direct comparisons to FISH and validate labeling of the target RNA in cells. As a biological context for testing the labeling method, stress granules were selected. In response to stress conditions, cells form non-membrane-bound cytosolic and nuclear RNA-protein assemblies to stall the translation of mRNA until the cells are no longer under stress. While most mRNAs can be concentrated to stress granules, different mRNAs have vastly distinct localization efficiencies. Using FISH, experiments indicate that the CDK6 mRNA is highly enriched in stress granules of mammalian cells CDK6 was used as a model system to fluorescently label and image the unique distribution pattern of the mRNA. Both the 6× and 1×MGA sequences were inserted at the 5′ end of the transcript, and the construct was placed under the control of the cytomegalovirus (CMV) promoter (FIG. 2A). Mouse neuroblastoma Neuro-2a cells were then transfected with these plasmids and incubated with 30 μM MGD2 for 20 min. The cells were then irradiated using 365 nm UV light to allow for covalent labeling of the aptamer. The media was replaced to washout excess dye, and cell stress conditions were induced by 45 min of arsenite exposure, a well characterized paradigm to induce formation of stress granules. Following fixation and immunofluorescence labeling of the stress granule marker protein (G3BP1), the cells were imaged using confocal microscopy.

In Neuro-2a cells exposed to arsenite stress, the formation of distinct cytoplasmic RNA granules was observed using 6×MGA-functionalized mCDK6 (6×MGA-mCDK6). In contrast, the signal from cells that were not exposed to arsenite was diffused uniformly throughout the cytoplasm, and no detectable stress granule enriched mCDK6 was observed. Encouraged by the ability to visualize RNA granules having six copies of the aptamer appended to the mRNA, imaging cells that were transfected with 1×MGA-functionalized mCDK6 was attempted (1×MGA-mCDK6). After similar arsenite treatment, RNA granules could be detected with a single copy of the aptamer fused to the mRNA. Moreover, when arsenite-treated cells were not UV-irradiated, no detectable foci formation was observed, indicating that RNA visualization is dependent on covalent attachment of the probe to the aptamer.

To further validate the specificity of this system, cells were transfected with CDK6 lacking the MGA sequence. The cells were incubated with MGD2 and UV irradiation was performed. These control cells did not show any labeling, indicating the absence of non-specific labeling of other cellular components. When comparing the stress granules detected with 1×MGA versus 6×MGA-functionalized mRNA, we did observe reduced fluorescence intensity in cytoplasmic mRNA granules. The difference was only ˜2-fold compared to the 6-fold smaller fusion of the 1×MGA construct, and the ability to detect RNA localization using a single aptamer fusion allows for minimal alteration of the target mRNA. Together, these results demonstrate that one is able to fluorescently label cellular mRNAs in a sequence-specific manner and observe their localization to stress granules.

Comparison of Fixed Cell Imaging of MGA/MGD2 with FISH

To validate that the fluorescence signal observed was arising from labeling of the target CDK6 mRNA, the Neuro-2a cells were simultaneously incubated with FISH probes complementary to the CDK6 sequence but bearing a spectrally orthogonal fluorophore. This experiment also enabled us to directly compare the sensitivity of the MGA/MGD2 system to the commonly used FISH technique for RNA labeling in fixed cells. Following arsenite stress, MGD2 labeling, and cell fixation, the cells were incubated with our custom FISH probes. Merged image analysis of the 1×MGA- and 6×MGA-labeled mRNA with the FISH signal showed an overlap of the fluorescence generated from these two approaches. Interestingly, some RNAs were observed by our MGA/MGD2 system that were not identified by FISH. This enhanced sensitivity was observed for both 1×MGA- and 6×MGA-functionalized mRNAs, indicating higher sensitivity for RNA detection than FISH. That may be because during FISH labeling, the fluorescent probe is hybridized after the RNA of interest is localized to granules. While this approach can identify ROIs that are spatially accessible to the FISH probes, other proteins and RNAs found within the granules compromise the ability of the probes to hybridize with the ROI. In contrast to FISH, the aptamer approach ensures that the MGA-functionalized mRNA is labeled with the fluorescent reporter before it is localized to the granules. This important distinction in the timing of RNA labeling allows for the detection of mRNA that otherwise would be inaccessible for FISH labeling.

Live Cell Imaging of mRNA

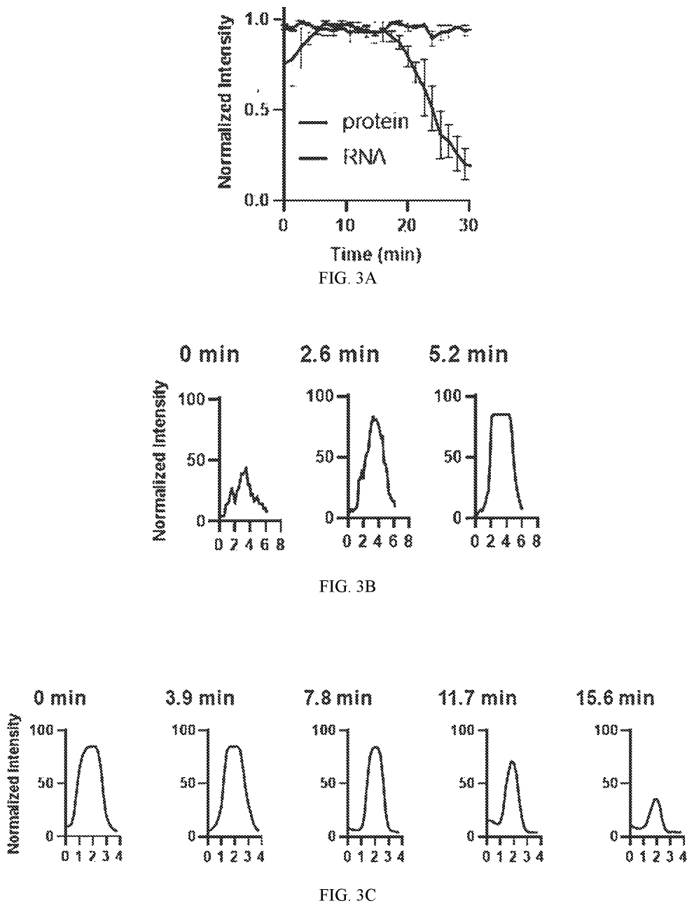

Experiments were performed to determine whether the MGD2/MGA system would be suitable for live cell imaging of endogenous mRNA localization and dynamics. Moreover, experiments were performed to determine whether one could simultaneously track the real-time localization properties of both RNA and proteins within a living cell. For this assay, Neuro-2a cells were co-transfected with expression plasmids for 1×MGA-mCDK6 and dual GFP-tagged G3BP1 protein. The expressed mRNA was labeled with MGD2 and photo crosslinked. The cells were then imaged to record the spatiotemporal features of both mRNA and protein in stress granules. After arsenite exposure, real-time confocal microscopy imaging of the granules revealed the dynamic nature of both the mRNA and the protein. Monitoring the signal intensity, the fluorescent signal generated from the MGD2 labeled granules was observed and remained consistent over >25 min of imaging. However, the GFP signal showed a sharp signal decrease after 17 min of imaging (FIG. 3A). Time-lapse imaging of the RNA granules also showed a gradual phase separation and maturation of RNA granules. These data highlight the ability of the aptamer approach to enable live-cell monitoring of RNA dynamics using only a single copy of the aptamer fusion and demonstrate the high photostability of the MG-aptamer pair.

After observing the motility and maturation of the granules, experiments were performed to determine whether one could also observe their dissolution. In mammalian cells, stress granules disassemble in the presence of small aliphatic molecules that disrupt weak hydrophobic interactions. Therefore, 1,6-hexanediol, an aliphatic alcohol commonly used for disassembly of stress granules was added. After arsenite-induced stress granule formation, the cells were incubated in 5% 1,6-hexanediol solution. The fluorescence signal of the labeled 1×MGA-mCDK6 was observed. In this experiment, signal from the protein granules disappeared after 7 min of incubation with 5% hexanediol. In contrast, most of the RNA granules exhibited a more sustained structural integrity, indicating that granules having high RNA content may be more resistant to dissolution (FIG. 3B). In some RNA granules, however, hexanediol triggered an observable dissolution of these phase-separated compartments (FIG. 3C). This observation indicates that the strength of intermolecular forces of the RNA granule components is heterogeneous across different granules. Therefore, these data indicate that the aptamer RNA labeling approach is able to validate assumptions of RNA properties.

### In Vitro Fluorescence Enhancement

A solution of 30 μM of MGD2 was mixed with 4 μM of the corresponding mRNA in 1×PBS (1.54 mM potassium monobasic, 155.17 mM sodium chloride, and 2.71 mM sodium phosphate dibasic with pH=7.2) and incubated at room temperature for 20 min. For comparison, a solution of MGD2 without any RNA was also prepared in 1×PBS. The fluorescence of these solutions was measured using a BioTek Cytation™ 5 plate reader with ex=620±20 nm and em=680±20 nm. The average fluorescence value from replicate experiments was used to calculate fluorescence enhancement.

### Cell Culture and Transfection

Neuro-2a cells were used for all cellular experiments. These cells were cultured in DMEM (high glucose) supplemented with 10% FBS at 37° C. in a humidified atmosphere of 5% CO₂ and 95% air. The cells were split every two days or once they reached >85% confluency. All cellular imaging was done on Cellview™ cell culture slides. All transfections were done using the Lipofectamine™ 3000 transfection reagent following the manufacturer recommended protocol with modifications: a solution of 5 μl of Opti-MEM™ media and 0.3 μl of Lipofectamine™ 3000 was mixed with a solution of 5 μL of Opti-MEM™, 0.3 μg of DNA, and 0.8 μL of P3000. This solution was incubated at room temperature for 20 min. The media from the cell culture wells was removed and the DNA Lipofectamine™ mixture was added directly to each chamber containing 60-80% confluent cells. Immediately after, 90 μL of 37° C. warmed media was added to each well for a total of 100 μL of solution. The cells were then placed back into the cell culture incubator for 12 h before conducting further experimentation.

1. A method comprising:
a) providing,
1) a conjugate comprising a fluorescent tag linked to a diazirine group and
2) a nucleic acid encoding RNA comprising a first aptamer segment that specifically binds the fluorescent tag and a second segment;
b) transfecting or inserting the nucleic acid into a cell such that RNA is expressed in the cell;
d) incubating the cell with the conjugate such that the fluorescent tag and the RNA form a complex inside the cell; and
e) exposing the complex to ultraviolet radiation such that a covalent bond between the RNA and the conjugate forms providing labeled RNA inside the cell with a fluorescent signal.

2. The method of claim 1 further comprising the step of imaging the fluorescent signal.

3. The method of claim 1 further comprising the step of tracking the location of the fluorescent signal.

4. The method of claim 1, wherein the second segment specifically binds to a protein in the cell.

5. The method of claim 1, wherein the first aptamer segment has a 5′ end consisting of the nucleic acid sequence of GGATCCCGACTGGCGAGAGCCAGGTAACGAATGGATCC (SEQ ID NO: 1).

6. The method of claim 5, wherein the conjugate is a salt of N-(4-((4-(dimethylamino)phenyl)(4-(3-(3-methyl-3H-diazirin-3-yl)propanamido)phenyl)methylene)cyclo hexa-2,5-dien-1-ylidene)-N-methylmethanaminium.

7. The method of claim 6, wherein exposing the complex to ultraviolet radiation is irradiation at 365 nm.

8. The method of claim 7, wherein exposing the complex to ultraviolet radiation is for not more than 15 min when the conjugate is at a concentration of 30 μM.

9. The method of claim 7, wherein exposing the complex to ultraviolet radiation is for not more than 10 min when the conjugate is at a concentration of 100 μM.

10. A method comprising:
a) providing,
1) a conjugate comprising a fluorescent tag linked to a diazirine group and
2) RNA comprising a first aptamer segment that specifically binds the fluorescent tag and a second segment;
b) transfecting or inserting the RNA into the cell;
c) incubating the cell with the conjugate such that the fluorescent tag and the RNA form a complex inside the cell; and
d) exposing the complex to ultraviolet radiation such that a covalent bond between the RNA and the conjugate forms providing labeled RNA inside the cell with a fluorescent signal.

11. The method of claim 10 further comprising the step of imaging the fluorescent signal.

12. The method of claim 10 further comprising the step of tracking the location of the fluorescent signal.

13. The method of claim 10, wherein the second segment specifically binds to a protein in the cell or hybridizes to nucleic acid in the cell.

14. The method of claim 10, wherein the first aptamer segment has a 5′ end consisting of the nucleic acid sequence of GGATCCCGACTGGCGAGAGCCAGGTAACGAATGGATCC (SEQ ID NO: 1).

15. The method of claim 14, wherein the conjugate is a salt of N-(4-((4-(dimethylamino)phenyl)(4-(3-(3-methyl-3H-diazirin-3-yl)propanamido)phenyl)methylene)cyclo hexa-2,5-dien-1-ylidene)-N-methylmethanaminium.

16. The method of claim 15, wherein exposing the complex to ultraviolet radiation is irradiation at 365 nm.

17. The method of claim 16, wherein exposing the complex to ultraviolet radiation is for not more than 15 min when the conjugate is at a concentration of 30 μM.

18. The method of claim 16, wherein exposing the complex to ultraviolet radiation is for not more than 10 min when the conjugate is at a concentration of 100 μM.

19. A salt of N-(4-((4-(dimethylamino)phenyl)(4-(3-(3-methyl-3H-diazirin-3-yl)propan amido) phenyl)methylene)cyclohexa-2,5-dien-1-ylidene)-N-methylmethanaminium.


SEQUENCE LISTING (8 sequences)

SEQ ID NO: 1 - DNA, 38 bp, Artificial
  Synthetic construct
ggatcccgactggcgagagccaggtaacgaatggatcc

SEQ ID NO: 2 - DNA, 424 bp, Artificial
  Synthetic construct
tctagatggtgttttggtttggtcaacggatcccgactggcgagagccaggtaacgaatggatccgttgacacccaacaaaaaaaaacacggatcccgactggcgagagccaggtaacgaatggatccgtgtaaaaaaaaacccaaaaagccgggatcccgactggcgagagccaggtaacgaatggatcccggcaaccaaccaaccaaccaaaaaggatcccgactggcgagagccaggtaacgaatggatcctttttccccagacaacgagagtcttggatcccgactggcgagagccaggtaacgaatggatccaagagagagaccacatgcaaaatctggatcccgactggcgagagccaggtaacgaatggatccagatgctagcgcggatccgaattcgagctccgtcgacaagcttt

SEQ ID NO: 3 - DNA, 32 bp, Artificial
  Synthetic construct
atatataccggtaccatggagaaggacagcct

SEQ ID NO: 4 - DNA, 32 bp, Artificial
  Synthetic construct
atatattctagaatcaggctgtgttcagctcc

SEQ ID NO: 5 - DNA, 43 bp, Artificial
  Synthetic construct
ctagcggatcccgactggcgagagccaggtaacgaatggatcc

SEQ ID NO: 6 - DNA, 43 bp, Artificial
  Synthetic construct
gatccggatccattcgttacctggctctcgccagtcgggatcc

SEQ ID NO: 7 - DNA, 32 bp, Artificial
  Synthetic construct
atatatgctagctagatggtgttttggtttgg

SEQ ID NO: 8 - DNA, 28 bp, Artificial
  Synthetic construct
atatatcttaagcgaattcggatccgcg